(19) United States
(12) Patent Application Publication
(10) Pub. No.: US 2015/0034093 A1
(43) Pub. Date: Feb. 5, 2015

(54) Method and Apparatus for Incrementally Repositioning the Mandible of a Patient

(71) Applicants: Bernard Michael Kruger, West Caldwell, NJ (US); Peter Kuch, Miami, FL (US); Edwyn Blanton Pyron, III, Boynton Beach, FL (US); Christopher Allen Rook, Wellington, FL (US)

(72) Inventors: Bernard Michael Kruger, West Caldwell, NJ (US); Peter Kuch, Miami, FL (US); Edwyn Blanton Pyron, III, Boynton Beach, FL (US); Christopher Allen Rook, Wellington, FL (US)

(21) Appl. No.: 13/958,230

(22) Filed: Aug. 2, 2013

(51) Int. Cl. A61F 5/56; A61C 7/36

(52) U.S. Cl. CPC A61F 5/566; A61C 7/36
USPC 128/848

(57) ABSTRACT

A method and apparatus for incrementally repositioning the mandible of a patient includes a mandible repositioning assembly having a support member defining opposed first and second female retention structures. The assembly includes first and second male retention members selectively coupled to the first and second female retention structures. In one embodiment, first and second sleeves may be received in respective female retention structures, each female retention structure defining an interior area configured to receive respective male retention members. Each male retention member includes a ball portion pivotally movable when coupled to a respective female retention structure and a mounting portion configured to mount to a patient's tooth. In use, respective male retention members may be coupled to respective female retention structures and to a selected dentition of the patient. A plurality of support members each having a different length may be interchanged so as to effectively reposition the patient's mandible.

### FIELD OF THE INVENTION

This invention relates generally to medical devices and, more particularly, to a fully adjustable dental appliance that incrementally repositions a patient's mandible for the treatment of temporomandibular joint dysfunction (“TMJ”), snoring, and sleep apnea disorders.

### BACKGROUND OF THE INVENTION

In the past, both fixed and removable dental appliances have been employed in dentistry to provide symptomatic relief for various head and neck disorders. Dental appliances which are removable have been used to treat conditions such as snoring, sleep apnea, bruxism, myofascial pain, TMD dysfunction and its associated pain. While several of these devices have been shown to be effective, they are, for the most, physiologically unacceptable and thereby negatively impact patient compliance. Since patient compliance is critical to the success of any treatment regimen, its lack thereof renders the proposed treatment ineffective.

It has been shown by Dawson, (1989 Evaluation, Diagnosis, and Treatment of Occlusal Problems (2^{nd} ed), Saint Louis, Mo., Mosby, pg. 184-190), that the placement of an occlusal splint thereby preventing the existing occlusion from dictating the jaw to jaw position in maximum intercuspation, may cause a relaxation of the muscles of mastication. This effect occurs because the teeth are separated—allowing the surrounding musculature to regain its own neuromuscular integrity. Even though splint construction may initiate a reduction in elevator muscle activity, in the long run violation of “freeway space” and an increase in vertical dimension beyond physiologic limits will cause an inevitable increase in elevator activity and pathological sequelae, such as the intrusion of the existing dentition may occur.

It has been shown by Weinberg and others, that eccentric placement of the mandibular condyle in glenoid fossa, as indicated in transcranial radiography, is frequently associated with an increase in TMD symptoms, and that by establishing concentricity of the condyle in the fossa symptoms may resolve. A majority of these eccentric displacements are posterior superior and by bringing the mandibular condyle down and forward along the protrusive path in a measured fashion is of paramount importance in the reestablishment of physiologic function.

Patients with TMD pathology are frequently treated with jaw repositioning devices in an attempt to regain proper physiologic function. Typical devices such as those mentioned in U.S. Pat. Nos. 5,085,584, 4,519,386, and 4,671,766, all attempt to disocclude the existing dentition, allow for neuromuscular deprogramming, and mandibular repositioning. The attempted corrections, however, are somewhat uncontrolled, are not reproducible, and come at the expense of a massive violation of the patients natural vertical dimension of occlusion.

The concept of mandibular/maxillary disarticulation in an attempt to relieve TMD symptoms is well known; however, the use of these devices to reduce pharyngeal airway obstruction and to treat sleep apnea or snoring is a relatively recent occurrence.

Sleep apnea is generally defined as cessation of airflow and breathing lasting 10 seconds or more. Hypoapnea refers to a 50% reduction for 10 seconds or more. Sleep apnea had been classified as central, obstructive, or mixed. With central sleep apnea (“CSA”), the respiratory musculature intermittently fails to function as the result of a Central Nervous System dysfunction (“CNS”), resulting in a decrease in oxygenation. In the case of obstructive sleep apnea (“OSA”), the respiratory musculature is functional but a blockage in the upper airway passages prevents normal inspiration. In mixed apnea the patient presents with a combination of the aforementioned problems.

Upper airway resistance syndrome (“UARS”) is characterized by loud snoring that causes frequent awakening. The UARS patient has all of the symptoms of OSA but without the hypoapneic sequellae. The snoring is generally caused by a relaxation of the pharyngeal musculature leading to airway restriction and causing a vibration of the surrounding tissue upon the passage of air. The tongue and soft palate may also be causative factors in snoring and OSA.

When the subject is in repose, the tongue may fall to the back of the throat thereby causing airway blockage. Controlled mandibular advancement widens the airway, and may reduce snoring.

Sleep apnea has been associated with hypertension, cardiovascular, and cerebrovascular disease. Mandibular advancement has been shown to reposition the tongue and open the airway, reducing the pathology associated with OSA, snoring, and UARS. Recent studies are indicating that parafunctional habits, such as bruxism and clenching, not only lead to severe occlusal wear, but also complicate oral appliance contraction. A number of oral appliances for the treatment of TMD and sleep apnea have been proposed to reposition the mandible. All of these devises move the jaw in a relatively uncontrolled fashion or cause a needless and destructive violation of the patients natural vertical dimension of occlusion. The proposed device, by contrast, allows the controlled and measured advancement of the mandible, allows for the movements to occur without disruption of normal condylar function, and does so in such a way as to minimize incursion on the patients normal vertical dimension of occlusion.

U.S. Pat. No. 5,427,117 discloses a dental device for the prevention of snoring and improving breathing. It includes an upper and lower arch attachment member of a deformable material in which a mold of the subjects maxillary and mandibular arch is formed. A post extends from the upper arch attachment member and contacts the lower arch attachment member so that the lower jaw is extended forward, thereby reducing snoring. The device is designed to permit some movement of the lower jaw for improved comfort, In a particular embodiment, the forward location of the post, with respect to the user, is adjustable to allow titration of the degree of mandibular advancement. In a preferred embodiment, the upper and lower jaws are “locked” such that they cannot be opened more than a predetermined amount. Translational or lateral jaw movements are restricted as well. U.S. Pat. Nos. 5,365,945, 5,566,683, 6,305,376, and 6,845,774 describe similar devices.

All of the aforementioned devices serve to advance the mandible in order to change pharyngeal anatomy by opening the airway. They do so at a price in that they do not respect the fixed posterior factors of mandibular movement, and they also cause a needless violation of Vertical Dimension of Occlusion, thereby causing unnecessary muscle pain and patient noncompliance. The posterior fixed factors include the shape and angulation of the glenoid fossa and the movement of the condyle along it (i.e. protrusive movement). The shape of the medial wall of the glenoid and the movement of the condyle along it (lateral Protrusion).

The shape of the superior portion of the glenoid fossa and the movement of the condyle along it (reciprocal movement). Intercondylar width, which affects the path of the mandible in lateral excursions. It must also be remembered that the mandibular condyle is an eccentric body and its center of rotation can only be determined by kinematic facebow application.

Therefore, it would be desirable to have a method and apparatus for repositioning the mandible of a dentistry patient that overcomes the limitations and disadvantages of the devices and patent described above. Further, it would be desirable to have a method and apparatus in which a dentist can repeatedly mount a mandible repositioning assembly to a selected dentition of a patient using a support member of different lengths in order to incrementally move the patient's mandible.

### SUMMARY OF THE INVENTION

A method and apparatus for incrementally repositioning the mandible of a patient according to the present invention includes a mandible repositioning assembly having a support member defining opposed first and second female retention structures. Further, the mandible repositioning assembly includes first and second male retention members that are selectively coupled to the first and second female retention structures in a pivotal engagement. In one embodiment, first and second sleeves may be received in respective female retention structures, each female retention structure defining an interior area configured to receive respective male retention members. Each male retention member includes a ball portion pivotally movable when coupled to a respective female retention structure and a mounting portion configured to mount to a patient's tooth. The male and female retention elements may be coupled together in a ball and socket configuration.

In use, respective male retention members may be coupled to respective female retention structures and to a selected dentition of the patient. A plurality of support members each having a different length may be selectively interchanged over time so as to effectively reposition the patient's mandible. For instance, respective ends of a particular support member may be mounted to a patient's maxillary first molar and the patient's mandibular bicuspid. The pivotal connections between the male and female retention members do not interfere with natural movement of the patient's mandibular condyles, the patient's natural vertical dimension of occlusion, and allows for superior, inferior, and lateral rotation of the mandible.

Using conventional radiological modalities, e.g. X-rays, a dentist may monitor ongoing repositioning and call for interchanging support members of different lengths to continue the mandibular repositioning as desired. This is what is referred to as “incremental repositioning.”

Therefore, a general object of this invention is to provide a mandibular repositioning assembly having a plurality of support members of various lengths along with male and female retention members that cooperate to mount a selected support member to a selected dentition of a patient, whereby to incrementally reposition the patient's mandible.

Another object of this invention is to provide a mandibular repositioning assembly in which opposite ends of a support member are selectively bonded to an upper tooth of a patient's dentition and to a lower tooth of the patient's dentition, respectively.

Still another object of this invention is to provide a mandibular repositioning assembly in which respective male and female retention structures are pivotally coupled together such that the mounted assembly minimizes incursion on the patient's normal vertical dimension of occlusion and does not interfere with the mandibular condyles natural movement.

Yet another object of this invention is to provide a mandibular repositioning assembly that allows for superior, inferior, and lateral rotation of the mandible.

A further object of this invention is to provide a mandibular repositioning assembly in which male and female retention structures are coupled and uncoupled in user-friendly snap fit configurations.

A still further object of this invention is to provide a method of repositioning a patient's mandible which includes mounting a mandibular repositioning assembly having a support member of one size to a selected dentition, removing said assembly after the mandible has been repositioned an intermediate amount, and then re-mounting the assembly using a support member of another size, and so on, until the mandible has been completely repositioned as desired.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a mandibular repositioning assembly according to a preferred embodiment of the present invention;

FIG. 2 is an exploded view of the assembly as in FIG. 1;

FIGS. 3a to 3c are side views of support members of differing lengths; and

FIG. 4 is a plan view of the assembly mounted to the teeth of an exemplary patient.

FIG. 5 is a perspective view of a mandibular repositioning assembly according to another embodiment of the present invention; and

FIG. 6 is an exploded view of the assembly as in FIG. 1.

### DESCRIPTION OF THE PREFERRED EMBODIMENT

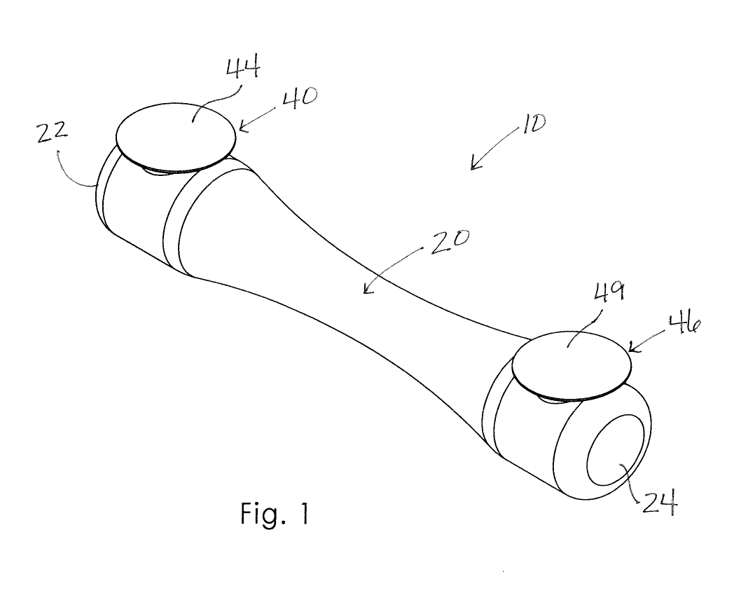

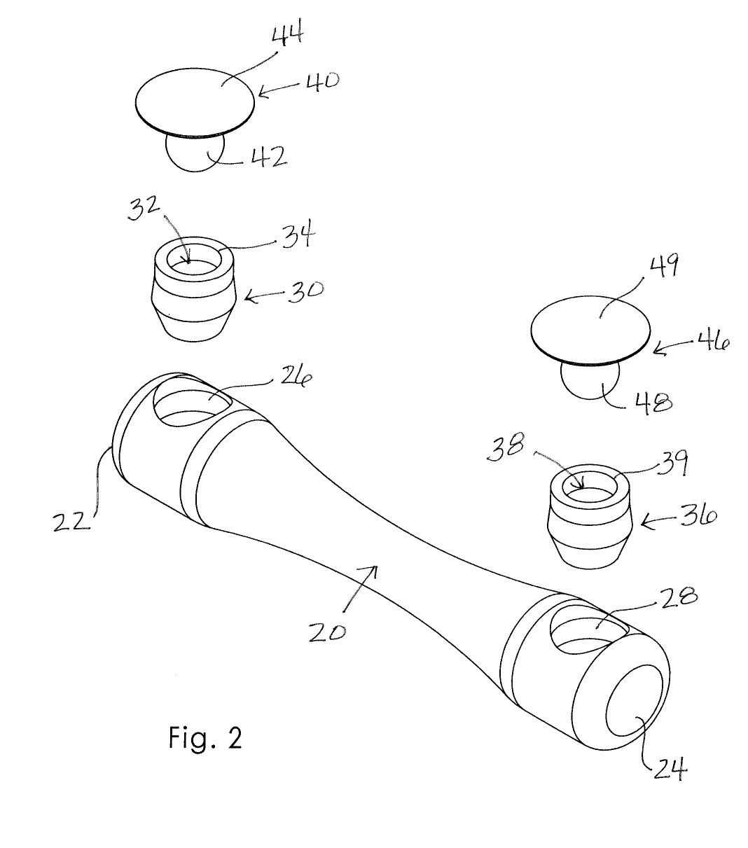

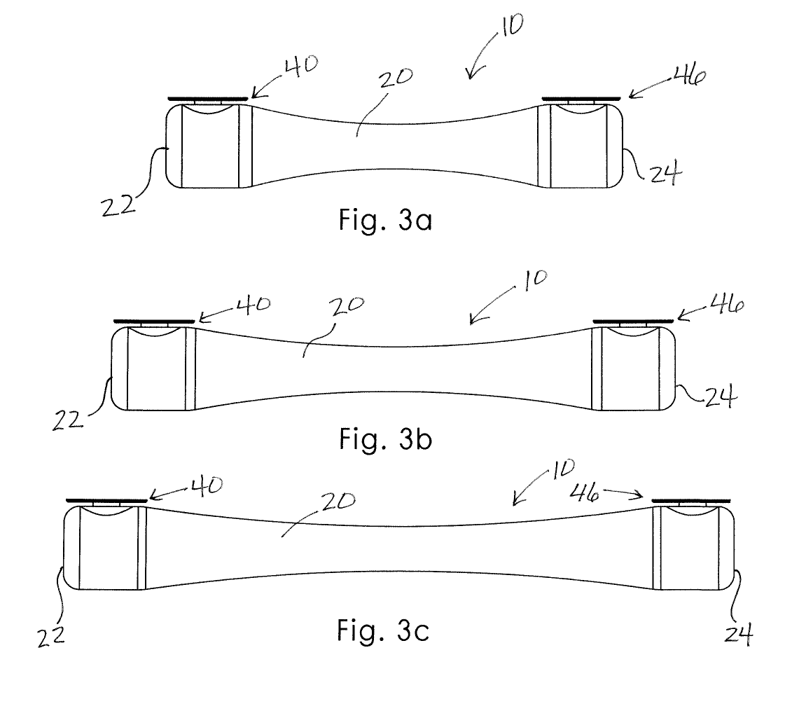

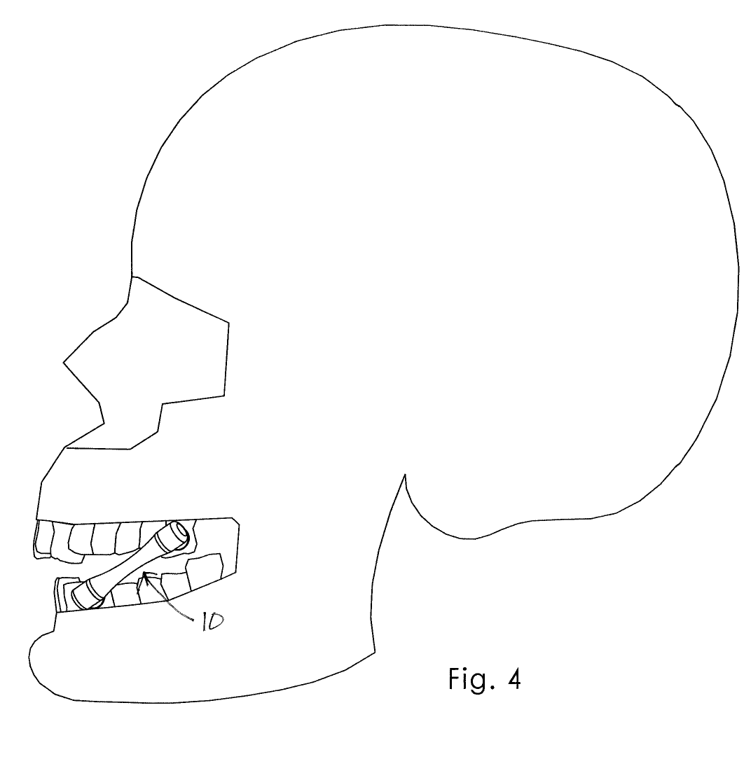

A method and assembly for incrementally repositioning the mandible of a dental patient will now be described in detail with reference to FIGS. 1 to 6 of the accompanying drawings. A mandibular repositioning assembly includes a plurality of support members, opposed female retention structures 26, 28 and a pair of male retention members 40, 46 that are mountable to a selected dentition of a patient.

The mandibular repositioning assembly 10 is provided for attachment to selected teeth of a patient for treatment of temporomandibular dysfunction (“TMD”), snoring, sleep apnea, and other head and neck disorders. The mandibular repositioning assembly 10 includes at least one support member 20 having opposed first 22 and second 24 ends. The support member 20 has a generally cylindrical configuration that extends in a linear direction. Preferably, the mandibular repositioning assembly 10 includes a plurality of support members 20, each support member 20 having a length that is different than a length of any other support member 20. Selective use of support members 20 of different lengths may be used successively so as to incrementally reposition a patient's mandible as will be discussed in more detail later. In one embodiment (not shown), a support member 20 may be length adjustable. More particularly, a single support member 20 may include an adjustment assembly that enables the length of the support member 20 to be either lengthened or shortened, as desired.

Each support member 20 defines a first female retention structure 26 and a second female retention structure 28 adjacent the first end 22 and second end 24 of a support member 20, respectively. Each female retention structure may be a bore having a generally cylindrical or ball-shaped interior area.

In one embodiment, the mandibular repositioning assembly 10 includes a first sleeve member 30 and a second sleeve member 36. The first female retention structure 26 and first sleeve member 30 have generally cylindrical configurations that are complementary such that the first sleeve member 30 is selectively received into the first female retention structure 26. Likewise, the second female retention structure 28 and second sleeve member 36 have generally cylindrical configurations that are complementary such that the second sleeve member 36 is selectively received into the second female retention structure 28. It is understood that even if the configurations described above are not perfectly cylindrical, they are nevertheless complementary to one another. Further, each of the first 30 and second 36 sleeves define open interior areas 32, 38 accessible through open tops 34, 39 respectively. First 40 and second 46 male retention members 40, 46 are selectively received into the interior areas 32, 38 as discussed below in more detail.

The first male retention member 40 also has a configuration complementary to that of the first female retention structure 26 and, in one embodiment, is selectively received therein. Likewise, the second male retention member 46 includes a configuration complementary to that of the second female retention structure 28 and, in one embodiment, is selectively received therein. In another embodiment, the first 40 and second 46 male retention members 40, 46 are selectively received into the interior areas 32, 38 defined by the first 30 and second 36 sleeves through the open tops 34, 39 thereof. It is understood that the male retention members are normally displaced from the support member 20 and are operatively coupled thereto according to a method of using the mandibular positioning assembly 10 as will be described later.

The first male retention member 40 includes a first ball portion 42 having a configuration that may be selectively received into the interior area of the first sleeve member 30. The first ball portion 42 is pivotal within the interior area 32 of the first sleeve member 30 and form a ball and socket arrangement. In one embodiment, the first ball portion 42 may be directly received into the first female retention structure 26 of the support member 20. Likewise, the second male retention member 46 includes a second ball portion 48 having a configuration that may be selectively received into the second sleeve member 36. The second ball portion 48 is pivotal within the interior area 38 of the second sleeve member 36 and forms a ball and socket arrangement. In one embodiment, the second ball portion 48 may be directly received into the second retention structure 28 of the support member 20.

Further, the first male retention member 40 includes a first mounting portion 44 coupled to the first ball portion 42 and having a generally planar configuration selectively mountable to a tooth of a patient. In other words, the first mounting portion 44 has a flat outer surface that may be adhered to a tooth of a dental patient, such as with a bonding material. Likewise, the second male retention member 46 includes a second mounting portion 49 coupled to the second ball portion 48 and having a generally planar configuration selectively mountable to a tooth of a patient. In other words, the second mounting portion 49 has a flat outer surface that may be adhered to a tooth of a dental patient, such as with a bonding material. In use, the first mounting portion 44 may be bonded to an upper tooth of a patient's dentition and the second mounting portion 49 may be bonded to a lower tooth of the patient's dentition as will be described in more detail below.

In one embodiment, the first ball portion 42 is a first ball screw and the first female retention structure 26 or first sleeve member 30 includes a threaded pattern complementary to that of the first ball screw. Likewise, in one embodiment, the second ball portion 48 is a second ball screw and the second female retention structure 28 or second sleeve member 36 includes an interior threaded pattern complementary to that of the second ball screw.

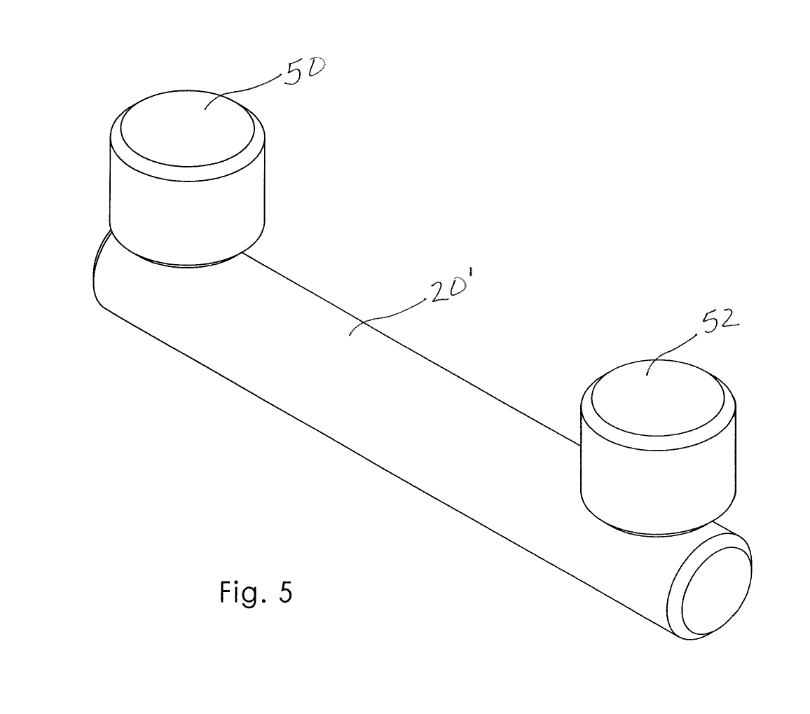

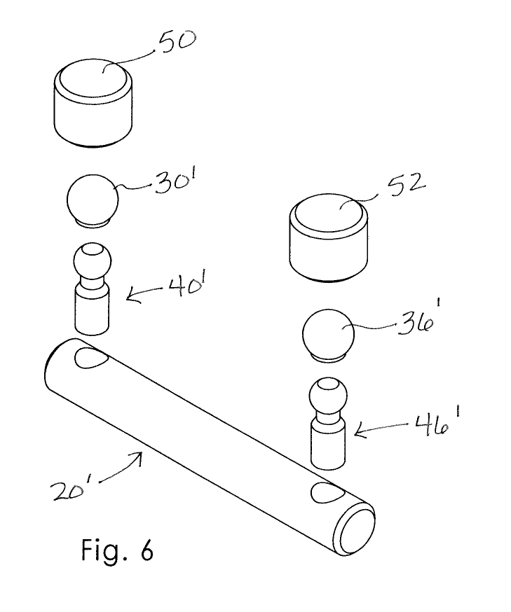

It is understood that the male and female retention members described above may be substantially reversed in some embodiments (FIGS. 5 and 6). In other words, the support member 20′ may include male retention members 40′, 46′ instead of defining female retention members and those male retention members may be received into female retention sleeves 30′, 36′ that are displaced from the support member 20. This embodiment may also include mounting structures 50, 52 having planar surfaces suitable for mounting to the teeth of a patient in a manner substantially similar to that described above.

The mandibular repositioning assembly 10 described above is used to reposition the mandible of a patient in a manner that does not unduly restrict the patient's vertical dimension of occlusion and that allows control of mandibular reclusion, and lateral excursion according to the steps described below.

The patient on whom the mandibular repositioning assembly 10 is to be installed is first placed in centric occlusion, i.e. the patient brings his teeth together. A distance is then determined between the patient's maxillary first molar and the patient's mandibular bicuspid. A support member 20 having a length generally equal to the determined distance between the selected teeth is selected for use in assembling the mandibular repositioning assembly 10. The male retention members 40, 46 are then coupled to respective female retention structures 26, 28 so that the dentist may confirm proper placement of the mandibular repositioning assembly 10. Preferably, the male retention members 40, 46 are engaged to the female retention structures 26, 28 or to the interior areas 32, 38 of respective sleeve members 30, 36 in a friction or snap fit configuration.

Once assembled, the proper position on the patient's selected dentition (i.e. the patient's maxillary first molar and the patient's mandibular bicuspid) may be determined. Specifically, an etching solution may be coated on respective male retention members 40, 46 which are then positioned on the selected dentition for a predetermined period of time. It is understood that this step will confirm that the support member 20 is the correct length and that the entire assembly 10 is configured appropriately to be mounted to the selected dentition. Positioning the coated first and second male retention members 40, 46 onto the selected dentition will leave appropriate indicator marks so as to prepare predetermined teeth to receive a mounting of the mandibular repositioning assembly 10. Then, the etching solution may be rinsed off of the selected dentition and from the male retention members 40, 46.

A bonding material may then be applied to the selected dentition of the patient, i.e. to the patient's maxillary first molar and to the patient's mandibular bicuspid. Then, the first 40 and second 46 male retention members may be mounted to the bonding material strategically placed on the outer surfaces of the particular teeth as indicated above.

Over time, such as a period of weeks, known radiologic modalities are expected to reveal movement of the mandible in a desired direction. In other words, X-rays may be used to determine if the patient's mandible has been repositioned in a desired direction. When desired progress has been made, the mandibular positioning assembly 10 may be removed from the dentition. The method may then be repeated, namely, determining a distance between the teeth to which the assembly 10 will again be mounted, determining an appropriate support member 20, and then re-mounting the assembly 10 to the selected dentition as described above. Over time, this method may be repeated again and again until the desired repositioning of the patient's mandible is complete.

Accordingly, the method and assembly described herein will enable a patient's mandible to be repositioned so as to successfully treat various head and neck conditions.

It is understood that while certain forms of this invention have been illustrated and described, it is not limited thereto except insofar as such limitations are included in the following claims and allowable functional equivalents thereof.

1. A mandibular repositioning assembly for selective attachment to teeth of a patient for the incremental repositioning of the patient's mandible as treatment of temporomandibular joint dysfunction (“TMD”), snoring, and sleep apnea disorders, comprising:
a support member having opposed first and second ends, said support member defining first and second female retention structures adjacent said first and said second ends, respectively; and
first and second male retention members operatively received in said first and said second female retention structures, respectively.

2. The mandibular repositioning assembly as in claim 1, wherein said first and said second male retention members have a configuration that is complementary to said first and said second female retention structures so as to be selectively received into said first and said second female retention structures, respectively, and to be pivotally movable therein.

3. The mandibular repositioning assembly as in claim 1, comprising:
first and second sleeve members selectively positioned in said first and said second female retention structures, respectively, said first and said second sleeve members each defining an interior area accessible through an open top, respectively;
wherein said first and said second male retention members have a configuration that is complementary to said open tops of said first and said second sleeves so as to be selectively received into said first and said second sleeves, respectively, and to be pivotally movable therein.

4. The mandibular repositioning assembly as in claim 3, wherein:
said first male retention member includes a first ball portion having a configuration selectively received into said first sleeve for pivotal movement therein and a first mounting portion having a generally planar configuration selectively mountable to a tooth of the patient;
said second male retention member includes a second ball portion having a configuration selectively received into said second sleeve for pivotal movement therein and a second mounting portion having a generally planar configuration selectively mountable to another tooth of the patient.

5. The mandibular repositioning assembly as in claim 1, wherein:
said first male retention member includes a first ball portion having a configuration selectively received into said first female retention structure for pivotal movement therein and a first mounting portion having a generally planar configuration selectively mountable to a tooth of the patient;
said second male retention member includes a second ball portion having a configuration selectively received into said second female retention structure for pivotal movement therein and a second mounting portion having a generally planar configuration selectively mountable to another tooth of the patient.

6. The mandibular repositioning assembly as in claim 1, wherein said support member includes a generally linear configuration.

7. The mandibular repositioning assembly as in claim 1, wherein respective male retention members and respective female retention structures are pivotally coupled together in a ball and socket configuration.

8. The mandibular repositioning assembly as in claim 3, wherein respective male retention members and respective interior areas of said sleeve members are pivotally coupled together in a ball and socket configuration.

9. The mandibular repositioning assembly as in claim 4, wherein:
said first ball portion is a first ball screw;
said first sleeve member has a threaded configuration so as to selectively receive said first ball screw;
said second ball portion is a second ball screw; and
said second sleeve member has a threaded configuration so as to selectively receive said second ball screw.

10. A method for incrementally repositioning the mandible of a patient in such a manner to not unduly restrict the patient's vertical dimension of occlusion and that allows control of mandibular protrusion, reclusion, and lateral excursion, said method including the steps of:
providing a mandible repositioning assembly having a support member defining opposed first and second female retention structures configured to selectively receive first and second male retention members in a pivotal engagement, respectively;
coupling said first and said second male retention members to said first and said second female retention members, respectively;
determining a desired positioning of said mandible repositioning assembly on a selected dentition of the patient;
applying a bonding material to the selected dentition of the patient; and
mounting said first and said second male retention members to said bonded material on the selected dentition and light curing into place.

11. The method of incrementally repositioning the mandible of a patient as in claim 10, comprising the steps:
coating an etching solution on said first and said second male retention members prior to mounting of said first and said second male retention members; and
positioning said coated first and second male retention members on the selected dentition of the patient for a predetermined amount of time.

12. The method of incrementally repositioning the mandible of a patient as in claim 10, comprising:
placing the patient in centric occlusion;
determining, prior to said step of mounting said first and said second male retention members, a distance between the patient's maxillary first molar and the patient's mandibular bicuspid; and
selecting said support member having a length substantially similar to said determined distance between the patient's maxillary first molar and the patient's mandibular bicuspid.

13. The method of incrementally repositioning the mandible of a patient as in claim 11, comprising rinsing said etching solution from said dentition and from said male retention members prior to mounting said male retention members.

14. The method of incrementally repositioning the mandible of a patient as in claim 12, wherein the step of mounting said first and said second male retention members includes mounting said first male retention member to the patient's maxillary first molar and mounting said second male retention member to the patient's mandibular bicuspid.

15. The method of incrementally repositioning the mandible of a patient as in claim 12, wherein said mandible repositioning assembly includes a plurality of support members each having a length different from the length of any other support member such that a respective support member is selected according to said determined distance between the patient's maxillary first molar and the patient's mandibular bicuspid.

16. The method of incrementally repositioning the mandible of a patient as in claim 15, further comprising:
removing said mandible repositioning assembly from the selected dentition;
replacing a respective support member of said removed mandible repositioning assembly with another support member having a length different than a length of said respective support member of said removed mandible repositioning assembly.

17. The method of incrementally repositioning the mandible of a patient as in claim 10, wherein said pivotal engagement between respective male and female retention structures allows for superior, inferior, and lateral rotation of the mandible of the patient, does not interfere with natural movement of the patient's mandibular condyles, and does not interfere with the patient's natural vertical dimension of occlusion.
